SEQUENCE LISTING (62 sequences)

SEQ ID NO: 1 - protein, 25 aa, Artificial Sequence
  NMU Agonist Peptide
XXXXXXXXXXXXXXXXXXFLFRPRN

SEQ ID NO: 2 - protein, 25 aa, Artificial Sequence
  NMU-25 Agonist
FRVDEEFQSPFASQSRGYFLFRPRN

SEQ ID NO: 3 - protein, 25 aa, Artificial Sequence
  NMU agonist
FRVDEEFQSPFASQSRGYFLFRPRN

SEQ ID NO: 4 - protein, 25 aa, Artificial Sequence
  NMU agonist
FRVDEEFQSPFASQSRGYFLFRPRN

SEQ ID NO: 5 - protein, 9 aa, Artificial Sequence
  NMU (17-25) Agonist
GYFLFRPRN

SEQ ID NO: 6 - protein, 25 aa, Artificial Sequence
  NMU agonist
FRVDEEFQSPFASQSRPYFLFRPRN

SEQ ID NO: 7 - protein, 25 aa, Artificial Sequence
  NMU Agonist
FRVDEEFQSPFASQSRGXXXXXXXX

SEQ ID NO: 8 - protein, 8 aa, Artificial Sequence
  NMU Agonist
XXXXXXXX

SEQ ID NO: 9 - protein, 7 aa, Artificial Sequence
  NMU Agonist
YFWRFRN

SEQ ID NO: 10 - protein, 8 aa, Artificial Sequence
  NMU Agonist
YFXWRPRN

SEQ ID NO: 11 - protein, 8 aa, Artificial Sequence
  NMU Agonist
YFGWRPRN

SEQ ID NO: 12 - protein, 8 aa, Artificial Sequence
  NMU Agonist
YFXWRPRN

SEQ ID NO: 13 - protein, 7 aa, Artificial Sequence
  NMU Agonist
FFWRPRN

SEQ ID NO: 14 - protein, 24 aa, Artificial Sequence
  NMU Agonist
FRVDEEFQSPFASQSRGYFWRPRN

SEQ ID NO: 15 - protein, 25 aa, Artificial Sequence
  NMU Agonist
FRVDEEFQSPFASQSRGYFXWRPRN

SEQ ID NO: 16 - protein, 8 aa, Artificial Sequence
  NMU Agonist
FWLFRPXN

SEQ ID NO: 17 - protein, 8 aa, Artificial Sequence
  NMU Agonist
FWLFRAXN

SEQ ID NO: 18 - protein, 8 aa, Artificial Sequence
  NMU Agonist
WFLFRPRX

SEQ ID NO: 19 - protein, 8 aa, Artificial Sequence
  NMU Agonist
FWLFRARN

SEQ ID NO: 20 - protein, 8 aa, Artificial Sequence
  NMU Agonist
WALFRARN

SEQ ID NO: 21 - protein, 8 aa, Artificial Sequence
  NMU Agonist
FALFRPRN

SEQ ID NO: 22 - protein, 25 aa, Artificial Sequence
  NMU Agonist
FRVDEEFQSPFASQSRGFWLFRPXN

SEQ ID NO: 23 - protein, 25 aa, Artificial Sequence
  NMU Agonist
FRVDEEFQSPFASQSRGFWLFRAXN

SEQ ID NO: 24 - protein, 25 aa, Artificial Sequence
  NMU Agonist
FRVDEEFQSPFASQSRGFWLFRPRX

SEQ ID NO: 25 - protein, 26 aa, Artificial Sequence
  NMU Agonist
CFRVDEEFQSPFASQSRGYFXWRPRN

SEQ ID NO: 26 - protein, 7 aa, Artificial Sequence
  NMU Agonist
FXWRPRN

SEQ ID NO: 27 - protein, 25 aa, Artificial Sequence
  NMU Agonist
XXXXXXXXXXXXXXXXXXXXXXXXX

SEQ ID NO: 28 - protein, 25 aa, Artificial Sequence
  NMU agonist
FRVDEEFQSPFASQSRPYFLFRPRN

SEQ ID NO: 29 - protein, 10 aa, Artificial Sequence
  NMU agonist
CGYFLFRPRN

SEQ ID NO: 30 - protein, 10 aa, artifical
  NMU agonist
CGYFLFRPRN

SEQ ID NO: 31 - protein, 10 aa, Artificial Sequence
  NMU agonist
CGYFLFRPRN

SEQ ID NO: 32 - protein, 10 aa, Artificial Sequence
  NMU agonist
CGYFLFRPRN

SEQ ID NO: 33 - protein, 26 aa, Artificial Sequence
  NMU agonist
CFRVDEEFQSPFASQSRGYFLFRPRN

SEQ ID NO: 34 - protein, 26 aa, Artificial Sequence
  NMU agonist
CFRVDEEFQSPFASQSRGYFLFRPRN

SEQ ID NO: 35 - protein, 26 aa, Artificial Sequence
  NMU agonist
CFRVDEEFQSPFASQSRGYFLFRPRN

SEQ ID NO: 36 - protein, 26 aa, Artificial Sequence
  NMU agonist
CFRVDEEFQSPFASQSRGYFLFRPRN

SEQ ID NO: 37 - protein, 26 aa, Artificial Sequence
  NMU agonist
CFRVDEEFQSPFASQSRGYFLFRPRN

SEQ ID NO: 38 - protein, 26 aa, Artificial Sequence
  NMU agonist
CFRVDEEFQSPFASQSRGYFLFRPRN

SEQ ID NO: 39 - protein, 26 aa, Artificial Sequence
  NMU agonist
CFRVDEEFQSPFASQSRGYFLFRPRN

SEQ ID NO: 40 - protein, 26 aa, Artificial Sequence
  NMU agonist
CFRVDEEFQSPFASQSRGYFXWRPRN

SEQ ID NO: 41 - protein, 26 aa, Artificial Sequence
  NMU agonist
CFRVDEEFQSPFASQSRGYFXWRPRN

SEQ ID NO: 42 - protein, 7 aa, Artificial Sequence
  NMU agonist
FLFRPRN

SEQ ID NO: 43 - protein, 7 aa, Artificial Sequence
  NMU agonist
FLFRPRN

SEQ ID NO: 44 - protein, 7 aa, Artificial Sequence
  NMU agonist
FLFRPRN

SEQ ID NO: 45 - protein, 15 aa, Artificial Sequence
  NMU agonist
CASQSRGYFLFRPRN

SEQ ID NO: 46 - protein, 15 aa, Artificial Sequence
  NMU agonist
CASQSRGYFLFRPRN

SEQ ID NO: 47 - protein, 15 aa, Artificial Sequence
  NMU agonist
CASQSRGYFLFRPRN

SEQ ID NO: 48 - protein, 20 aa, Artificial Sequence
  NMU agonist
CFQSPFASQSRGYFLFRPRN

SEQ ID NO: 49 - protein, 20 aa, Artificial Sequence
  NMU agonist
CFQSPFASQSRGYFLFRPRN

SEQ ID NO: 50 - protein, 20 aa, Artificial Sequence
  NMU agonist
CFQSPFASQSRGYFLFRPRN

SEQ ID NO: 51 - protein, 27 aa, Artificial Sequence
  NMU agonist
CCFRVDEEFQSPFASQSRGYFLFRPRN

SEQ ID NO: 52 - protein, 27 aa, Artificial Sequence
  NMU agonist
CCFRVDEEFQSPFASQSRGYFLFRPRN

SEQ ID NO: 53 - protein, 27 aa, Artificial Sequence
  NMU agonist
CCFRVDEEFQSPFASQSRGYFLFRPRN

SEQ ID NO: 54 - protein, 26 aa, Artificial Sequence
  NMU agonist
CFRVDEEFQSPFASQSRGYFLFRPRN

SEQ ID NO: 55 - protein, 26 aa, Artificial Sequence
  NMU agonist
CFRVDEEFQSPFASQSRGYFLFRPRN

SEQ ID NO: 56 - protein, 26 aa, Artificial Sequence
  NMU agonist
CFRVDEEFQSPFASQSRGYFLFRPRN

SEQ ID NO: 57 - protein, 26 aa, Artificial Sequence
  NMU agonist
CFRVDEEFQSPFASQSRGYFLFRPRN

SEQ ID NO: 58 - protein, 9 aa, Artificial Sequence
  NMU agonist
GYFLFRPRN

SEQ ID NO: 59 - protein, 9 aa, Artificial Sequence
  NMU agonist
GYFLFRPRN

SEQ ID NO: 60 - protein, 9 aa, Artificial Sequence
  NMU agonist
GYFLFRPRN

SEQ ID NO: 61 - protein, 26 aa, Artificial Sequence
  NMU agonist
CFRVDEEFQSPFASQSRPYFLFRPRN

SEQ ID NO: 62 - protein, 26 aa, Artificial Sequence
  NMU agonist
CFRVDEEFQSPFASQSRPYFLFRPRN

(19) United States
(12) Patent Application Publication
(10) Pub. No.: US 2011/0294735 A1
(43) Pub. Date: Dec. 1, 2011

(54) MECHANISM OF NEUROMEDIN U ACTION AND USES THEREOF

(75) Inventors: Donald J. Marsh, Hillsborough, NJ (US); Andrea M. Peier, Berkeley Heights, NJ (US); Alessandro Pocai, Westfield, NJ (US)

(73) Assignee: MERCK SHARP & DOHME CORP., Rahway, NJ (US)

(21) Appl. No.: 13/122,783

(22) PCT Filed: Oct. 30, 2009
(86) PCT No.: PCT/US09/62660

§ 371 Date: Apr. 6, 2011

Related U.S. Application Data
(60) Provisional application No. 61/198,337, filed on Nov. 5, 2008.

(51) Int. Cl. A61K 38/16; G01N 30/00; A61P 3/04; A61P 3/10; G01N 33/74; A61P 3/00

(52) U.S. Cl. 514/11.7; 435/7.92; 436/501; 514/9.7; 73/61.52

(57) ABSTRACT

The use of neuromedin U receptor agonists to elevate the levels of GLP-1 and/or PYY in an individual in need of an increase in its levels of GLP-1 and/or PYY is described. Further described is the use of neuromedin U receptor agonists to lower the levels of glucagon in an individual in need of lowered glucagon levels. Thus, methods for elevating GLP-1 and/or PYY and lowering glucagon levels in an individual by administering to the individual compositions comprising a neuromedin U receptor agonist and optionally one or more dipeptidyl peptidase IV (DPP-IV) inhibitors are described. In light of the ability of NMU receptor agonists to raise GLP-1 and PYY levels and lower glucagon levels post-administration, methods are described for evaluating the efficacy of a treatment regimen for a metabolic disorder that includes administering a composition comprising a neuromedin U receptor agonist to an individual comprising measuring the level of glucagon-like peptide 1 (GLP-1) and/or peptide YY (PYY) and/or glucagon in the individual before, during, and after the treatment regimen.

### CROSS REFERENCE TO RELATED APPLICATIONS

Not Applicable

### BACKGROUND OF THE INVENTION

(1) Field of the Invention

The present invention relates to the use of neuromedin U receptor agonists to elevate the levels of GLP-1 and/or PYY in an individual in need of an increase in its levels of GLP-1 and/or PYY. The present invention further relates to the use of neuromedin U receptor agonists to lower the levels of glucagon in an individual in need of lowered glucagon levels. Thus, the present invention relates to methods for elevating GLP-1 and/or PYY and lowering glucagon levels in an individual by administering to the individual compositions comprising a neuromedin U receptor agonist and optionally one or more dipeptidyl peptidase IV (DPP-IV) inhibitors. The present invention farther relates to methods for evaluating the efficacy of a treatment regimen for a metabolic disorder that includes administering a composition comprising a neuromedin U receptor agonist to an individual comprising measuring the level of glucagon-like peptide 1 (GLP-1) and/or peptide YY (PYY) and/or glucagon in the individual before, during, and after the treatment regimen.

(2) Description of Related Art

Neuromedin U (NMU) was originally isolated from porcine spinal cord based upon its ability to contract rat uterine smooth muscle and has since been implicated in a variety of other physiological processes, including stress, nociception, inflammation, cardiovascular function and energy homeostasis. Characterization of NMU has identified three peptides with similar bioactivity, full length NMU, (a 25-mer (NMU-25)) in humans, pigs, and dogs, a 23-mer (NMU-23) in rats and mice, and an 8-mer (NMU-8). NMU-8 is derived from cleavage of full-length NMU and shares an identical C-terminus with the full-length precursor. NMU-8 is highly conserved among vertebrates, containing seven C-terminal residues that are identical across all species that have been examined; these residues are critical for bioactivity (Brighton et al., Pharmacol. Rev. 56: 231-248 (2004)).

NMU's role in the regulation of energy homeostasis is supported by both pharmacologic and genetic data. Properties of NMU include inhibition of food intake and increase in energy expenditure seen when the substance is administered centrally (Howard et al., Nature 406: 70-74 (2000); Nakazato et al., Biochem. Biophys. Res. Comm. 277: 191-194 (2000); Ivanov et al., Endocrinol. 143: 3813-3821 (2002); and Wren et al., Endocrinol., 143: 4227-4234 (2002)). NMU-deficient mice develop obesity characterized by hyperphagia and reduced energy expenditure (Hanada et al., Nat. Med., 10: 1067-1073 (2004)), and transgenic mice overexpressing NMU are lean and hypophagic (Kowalski et al., J. Endocrinol. 185: 151-164 (2005)). The internal energy status of an animal affects expression and release of NMU as well (Wren et al., ibid.).

Two high affinity NMU receptors, NMUR1 (Intl. Patent Appl. No. PCT/US99/15941) and NMUR2 (U.S. Pat. No. 7,163,799), have been identified. NMUR1 is predominantly expressed in the periphery, whereas NMUR2 is primarily expressed in the brain. Pharmacologic experiments have served to better define NMU's short- and long-term effects on energy homeostasis and to identify which NMU receptor(s) are involved in mediating these actions. It has been shown that acute administrations of NMU either centrally or peripherally reduce food intake in mice in a dose-dependent fashion. The anorectic actions of centrally administered NMU are absent in NMUR2-deficient (Nmur2^{-/-}) mice but are present in NMUR1-deficient (Nmur1^{-/-}) mice. In contrast, the anorectic actions of peripherally administered NMU are absent in Nmur1^{-/-} mice and present in Nmur2^{-/-} mice. Additionally, acute peripheral administration of NMU dose-dependently increases core body temperature in mice, suggesting that NMUR1 may also modulate energy expenditure. Chronic administration of NMU either centrally or peripherally reduces food intake, body weight and adiposity in mice, again in a dose-dependent fashion. In Nmur2^{-/-} transgenic mice, body weight, body composition, body temperature and food intake are largely unaffected by chronic central administration of rat NMU-23. In Nmur1^{-/-} transgenic mice, body weight, body composition and food intake are largely unaffected by chronic peripheral administration of rat NMU-23.

Published International Application No. WO2007/109135 discloses that both NMUR1- and NMUR2-selective agonists and NMUR1/2 non-selective agonists are useful for the treatment of metabolic disorders such as obesity. However, while NMU and its analogs are useful for treating metabolic disorders, there is always a need for new methods for evaluating the efficacy of a treatment comprising NMU or analog thereof.

### BRIEF SUMMARY OF THE INVENTION

The present invention provides for the use of neuromedin U receptor agonists to elevate or enhance the levels of GLP-1 and/or PYY in an individual in need of an increase in its levels of GLP-1 and/or PYY. The present invention further provides for the use of neuromedin U receptor agonists to lower the levels of glucagon in an individual in need of lowered glucagon levels. Thus, the present invention provides methods for elevating GLP-1 and/or PYY and/or lowering glucagon levels in an individual by administering to the individual compositions comprising a neuromedin U receptor agonist and optionally one or more dipeptidyl peptidase IV (DPP-IV) inhibitors. The present invention further provides methods for evaluating the efficacy of a treatment regimen for a metabolic disorder that includes administering a composition comprising a neuromedin U receptor agonist to an individual comprising measuring the level of glucagon-like peptide 1 (GLP-1) and/or peptide YY (PYY) and/or glucagon in the individual before, during, and after the treatment regimen.

Therefore, the present invention provides methods for elevating or enhancing GLP-1 and/or PYY levels in an individual in which there is a need to elevate or enhance the levels of GLP-1 and/or PYY in the individual comprising providing to the individual a therapeutically effective amount of a composition comprising a neuromedin U receptor agonist. Further, provided are methods for lowering or reducing glucagon levels in an individual in which there is a need to lower or reduce the levels of glucagon in the individual comprising providing to the individual a therapeutically effective amount of a composition comprising a neuromedin. U receptor agonist. Further still, methods are provided for elevating or enhancing GLP-1 and/or PYY levels while simultaneously lowering or reducing glucagon levels in an individual in which there is a need to simultaneously elevate or enhance the levels of GLP-1 and/or PYY and lower or reduce the glucagon levels in the individual comprising providing to the individual a therapeutically effective amount of a composition comprising a neuromedin U receptor agonist.

A therapeutically effective amount of a neuromedin U receptor agonist is that amount which elevates or enhances the level of GLP-1 and/or PYY and/or lowers or reduces the level of glucagon in the plasma of an individual over the basal levels of GLP-1 and/or PYY and/or glucagon in the individual prior to administration of the neuromedin U receptor agonist to the individual. In particular embodiments, the composition will further include one or more dipeptidyl peptidase IV (DPP-IV) inhibitors. Examples of DPP-IV inhibitors include but are not limited to, isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, and SK-0403 and pharmaceutically acceptable salts thereof.

In embodiments wherein the neuromedin U receptor agonist is neuromedin U or analog thereof, the neuromedin U receptor agonist will have the formula

Z¹-peptide-Z²

wherein the peptide has the amino acid sequence X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵ (SEQ ID NO:27), wherein amino acids 1 to 17 can be any amino acid or absent; wherein amino acid X¹⁸ is absent, Y, W, F, a des-amino acid or an acyl group; amino acid X¹⁹ is A, W, Y, F or an aliphatic amino acid; amino acid X²⁰ is absent, L, G, sarcosine (Sar), D-Leu, NMe-Leu, D-Ala or A; amino acid X²¹ is F, NMe-Phe, an aliphatic amino acid, an aromatic amino acid, A or W; X²² is R, K, A or L; amino acid X²³ is P, Sar, A or L; amino acid X²⁴ is R, Harg or K; and amino acid X²⁵ is N, any D- or L-amino acid, Nle or D-Nle, A.; and Z¹ is an optionally present protecting group that, if present, is joined to the N-terminal amino group; and Z² is NH₂ or an optionally present protecting group that, if present, is joined to the C-terminal carboxy group, and pharmaceutically acceptable salts thereof.

The present invention further provides a method of determining the efficacy of a composition comprising a neuromedin U receptor agonist given to an individual for the treatment of a metabolic disorder, comprising (a) assaying a plasma sample from the individual to determine a level of GLP-1 and/or PYY and/or glucagon at a first time point; (b) administering the composition to the individual; and (c) thereafter assaying a plasma sample from the individual to determine the level of GLP-1 and/or PYY and/or glucagon at a second time point; wherein an increased level of GLP-1 and/or PYY and/or decreased level of glucagon at the second time point relative to the first time point is indicative of the efficacy of the composition in treating the metabolic disorder.

In a further embodiment, provided is a method for determining the efficacy of a neuromedin U receptor agonist-based therapeutic regime being administered to an individual to alleviate a metabolic disorder, comprising (a) assaying a plasma sample from an individual to determine a level of GLP-1 and/or PYY and/or glucagon at a first time point; (b) assaying a second plasma sample from the individual to determine a level of GLP-1 and/or PYY and/or glucagon at a second time point, wherein the therapeutic regime is followed by the individual between the first time point and the second time point; and (c) comparing the level at the second time point to the level determined in (a), wherein a change in the GLP-1 and/or PYY and/or glucagon levels compared to basal levels of the GLP-1 and/or PYY and/or glucagon in the individual prior to administration of the neuromedin U agonist at the second time point is an indication of the efficacy of the therapeutic regime.

In a further still embodiment, provided is a method for determining the appropriate dosage of a composition comprising a neuromedin U receptor agonist given to an individual for the treatment of a metabolic disorder, comprising (a) assaying a plasma sample from the individual to determine a level of GLP-1 and/or PYY and/or glucagon at a first time point; (b) administering the composition to the individual; (c) thereafter assaying a plasma sample from the individual to determine the level of GLP-1 and/or PYY and/or glucagon at a second time point; (d) determining whether the composition was administered at the appropriate dosage, wherein an increased level of GLP-1 and/or PYY and/or decreased level of glucagon at the second time point relative to the first time point is indicative of the efficacy of the composition in treating the metabolic disorder at the dosage administered; and (e) adjusting dosage as needed.

As used throughout the specification and in the appended claims, the singular forms “a”, “an”, and “the” include plural reference unless the context clearly dictates otherwise.

As used herein, the term “individual” encompasses any mammal, including but not limited to, humans, rodents such as the rat or mouse, dogs, and primates.

As used herein, “therapeutic regime” refers to any course of therapy prescribed or recommended by a physician or veterinarian or followed by an individual for the treatment or control of obesity, wherein the course of therapy includes the administration of at least one appetite suppressant. The therapeutic regime may include combination treatment with more than one active pharmaceutical compound or may be the administration of a single appetite suppressant drug. The therapeutic regime may further include other methods of treatment such as diet and exercise, in accordance with a physician or veterinarian recommended treatment plan or a treatment plan proposed by the individual.

As used herein, “appropriate dosage” refers to the dosage of a known pharmaceutical compound or test compound at which the compound is efficacious in suppressing appetite or inducing satiety. The appropriate dosage may vary with a variety of factors including the species and weight of the individual and the class of compound.

“Metabolic disorders” include, but are not limited to, obesity, metabolic syndrome or syndrome X, type II diabetes, complications of diabetes such as retinopathy, hypertension, dyslipidemias, cardiovascular disease, gallstones, osteoarthritis, and certain forms of cancers. The obesity-related disorders herein are associated with, caused by, or result from obesity.

“Obesity” is a condition in which there is an excess of body fat. The operational definition of obesity is based on the Body Mass Index (BMI), calculated as body weight per height in meters squared (kg/m2). “Obesity” refers to a condition whereby an otherwise healthy individual has a Body Mass Index (BMI) greater than or equal to 30 kg/m², or a condition whereby an individual with at least one co-morbidity has a BMI greater than or equal to 27 kg/m2. An “obese individual” is an otherwise healthy individual with a Body Mass Index (BMI) greater than or equal to 30 kg/m² or an individual with at least one co-morbidity with a BMI greater than or equal to 27 kg/m². An “individual at risk for obesity” is an otherwise healthy individual with a BMI of 25 kg/m² to less than 30 kg/m² or an individual with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby an individual with at least one obesity-induced or obesity-related co-morbidity that requires weight reduction or that would be improved by weight reduction, has a BMI greater than or equal to 25 kg/m². In Asian countries, including Japan, an “obese individual” refers to a subject with at least one obesity-induced or obesity-related co-morbidity that requires weight reduction or that would be improved by weight reduction, with a BMI greater than or equal to 25 kg/m2. In Asian countries, an “individual at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of a compound to reduce or maintain the body weight of an obese individual. One outcome of treatment may be reducing the body weight of an obese subject relative to that individual's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the individual, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of a compound to reduce or maintain the body weight of an individual at risk of obesity. One outcome of prevention may be reducing the body weight of an individual at risk of obesity relative to that individual's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in an individual at risk of obesity. Another outcome of prevention may be decreasing the occurrence and/or severity of obesity-related disorders if the treatment is administered prior to the onset of obesity in an individual at risk of obesity. Moreover, if treatment is commenced in already obese individuals, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient individuals, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer.

The term “diabetes,” as used herein, includes both insulin-dependent diabetes mellitus (IDDM, also known as type I diabetes) and non-insulin-dependent diabetes mellitus (NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese.

The term “individual” is meant to include humans and companion or domesticated animals such as dogs, cats, horses, and the like. Therefore, the compositions comprising formula I are also useful for treating or preventing obesity and obesity-related disorders in cats and dogs. As such, the term “mammal” includes companion animals such as cats and dogs.

The term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration. The neuromedin U receptor agonist may be in multimers (for example, heterodimers or homodimers) or complexes with itself or other peptides. As a result, pharmaceutical compositions of the invention may comprise one or more neuromedin U receptor agonists in such multimeric or complexed form.

### BRIEF DESCRIPTION OF THE DRAWINGS

FIG. 1A shows that acute peripheral administration of NMU increases plasma levels of PYY in obese mice: 10 mpk of NMU increased PYY levels at four-hours post dose.

FIG. 1B shows that 10 mpk of NMU increased PYY levels at two-hours and four-hours post dose.

FIG. 2A shows that acute peripheral administration of NMU increased plasma levels of total GLP-1 in obese mice: 10 mpk of NMU increased GLP-1 levels at four-hours post dose.

FIG. 2B shows that chronic treatment of NMU (3 mpk/day) given subcutaneously for two weeks led to a significant increase in total GLP-1.

FIG. 3 shows that changes in GLP-1 levels are mediated by NMUR1.

FIG. 4A shows that acute peripheral administration of the PEGylated NMU analog NMU12 increased plasma levels of PYY in obese mice: the NMU12 analog dose-dependently increased PYY levels in plasma 18-hours post dose in the presence of food.

FIG. 4B shows that in the absence of food, 10 mpk of NMU12 did not increase PYY levels 18-hr post dose.

FIG. 5A shows that acute peripheral administration of NMU12 increased plasma levels of total GLP-1 in obese mice: 10 mpk of NMU12 increased GLP-1 levels at 18-hours post dose in the presence of food.

FIG. 5B shows that 10 mpk of NMU12 increased GLP-1 levels at 18-hours post dose in the absence of food.

FIG. 6A shows that 0.1 mpk and 1 mpk of NMU12 reduced plasma glucose levels 15 minutes after an oral glucose tolerance test.

FIG. 6B shows that 0.1 mpk and 1 mpk of NMU12 reduced glucagon levels 15 minutes after an oral glucose tolerance test.

### DETAILED DESCRIPTION OF THE INVENTION

The inventors have discovered that neuromedin U receptor agonists effect an increase in GLP-1 and PYY levels over basal levels in an individual within a short period of time following administration of the agonist to the individual. A decrease in the levels of glucagon was also observed. The effect of neuromedin U receptor agonists on GLP-1, PYY, and glucagon levels can be observed in the individual in as little as two hours post-administration.

In general, GLP-1 secretion by L cells is dependent on the presence of nutrients in the lumen of the small intestine. The secretagogues (agents that cause or stimulate secretion) of this hormone include major nutrients like carbohydrate, protein and lipid. GLP-1 effects an increase in insulin secretion from the pancreas in a glucose-dependent manner, an increase in beta cells mass and insulin gene expression, an inhibition in acid secretion, a delay in gastric emptying in the stomach, a decrease in food intake by increasing satiety, and a decrease glucagon secretion from the pancreas. Glucagon is synthesized and secreted from alpha cells (α-cells) of the islets of Langerhans, which are located in the endocrine portion of the pancreas. In rodents, the alpha cells are located in the outer rim of the islet whereas in humans, islet structure is much less segregated and the alpha cells are distributed throughout the islet. In general, secretion of glucagon is caused by a decrease in plasma glucose. The effect of glucagon is to raise glucose levels in the blood. PYY exerts its action through NPY receptors, inhibits gastric motility and increases water and electrolyte absorption in the colon. PYY may also suppress pancreatic secretion. It is secreted by the neuroendocrine cells in the ileum and colon in response to a meal, and has been shown to reduce appetite. Leptin also reduces appetite in response to feeding, but obese people develop a resistance to leptin and obese people secrete less PYY than normal people; however, in response to added PYY, obese people will reduce their food intake.

Thus, the significance of elevating GLP-1 and/or PYY and/or decreasing glucagon levels in an individual to treat a metabolic disorder such as diabetes or obesity are well known in the scientific and medical communities (See for example, Hoist et al., Trends Mol. Med. 14: 161-8 (2008); Hoist, Physiol. Rev. 87: 1409-39 (2007); Ueno et al., Regul. Pept. 145: 12-6 (2007)). Various means for enhancing the levels of GLP-1 and/or PYY and/or lowering the levels of glucagon or mimicking the therapeutic effect of GLP-1 and/or PYY or antagonizing glucagon action have been the object of a large number of patents and applications for patents.

Therefore, in light of the above discovery, the present invention provides for the use of neuromedin U receptor agonists to elevate or enhance the levels of GLP-1 and/or PYY in an individual in need of an increase in its levels of GLP-1 and/or PYY. The present invention further provides for the use of neuromedin U receptor agonists to lower or reduce the levels of glucagon in an individual in need of lowered glucagon levels. In general, these two effects will occur simultaneously in the individual post-administration of the agonist, therefore, provided is the use of neuromedin U receptor agonists to elevate or enhance the levels of GLP-1 and/or PYY in an individual while effecting a decrease or reduction in the levels of glucagon in the individual. Thus, the present invention provides methods for elevating or enhancing GLP-1 and/or PYY levels and lowering or reducing glucagon levels in an individual by administering to the individual compositions comprising a neuromedin U receptor agonist and optionally one or more dipeptidyl peptidase IV (DPP-IV) inhibitors.

The present invention further provides methods for evaluating the efficacy of a treatment regimen for a metabolic disorder that includes administering a composition comprising a neuromedin U receptor agonist to an individual comprising measuring the level of glucagon-like peptide 1 (GLP-1) and/or peptide YY (PYY) and/or glucagon in the individual before, during, and after the treatment regimen. For example, using the methods disclosed herein, establishment of the efficacy of a composition comprising a neuromedin U receptor agonist in a treatment of a metabolic disorder can be made within several hours of administration of the composition. This can significantly reduce the amount of time necessary to eliminate non-efficacious compositions or dosages from a treatment regime. Consequently, the method enables an efficacious treatment regime for a metabolic disorder to be designed for an individual in a short period of time. The method facilitates routine monitoring of a therapeutic regime administered to an individual, which permits the dosage of the composition comprising the neuromedin U receptor agonist to be adjusted as needed to maintain a particular level of GLP-1 and/or PYY and/or glucagon in the individual undergoing the treatment and thus maintain a particular level of efficacy for the treatment regime. In addition, in drug development, the methods herein save resources and funds from being spent on compositions or treatment regimes that are not efficacious in elevating GLP-1 and/or PYY levels and/or decreasing glucagon levels in an individual.

Therefore, provided is a method for determining the efficacy of a composition comprising a neuromedin U receptor agonist given to an individual for the treatment of a metabolic disorder, for example, diabetes or obesity. The method involves the following steps. A plasma sample from the individual is assayed to determine a level of GLP-1 and/or PYY and/or glucagon at a first time point. Then, the composition comprising the neuromedin U receptor agonist is administered to the individual. At a second time point following administration of the composition, a plasma sample from the individual is obtained and the level of GLP-1 and/or PYY and/or glucagon determined. An increased level of GLP-1 and/or PYY and/or a decrease of glucagon at the second time point relative to the level at the first time point is indicative that the composition is efficacious in treating the metabolic disorder.

The above method can be used to monitor the effectiveness of a dosing regime involving use of a composition comprising a neuromedin U agonist for a metabolic disorder (e.g., diabetes or obesity) over time as follows. A plasma sample from the individual is assayed to determine a level of GLP-1 and/or PYY and/or glucagon at a first time point. Then, the composition comprising the neuromedin U receptor agonist is administered to the individual. At a second time point following administration of the composition, a plasma sample from the individual is obtained and the level of GLP-1 and/or PYY and/or glucagon determined. An increased level of GLP-1 and/or PYY and/or decreased level of glucagon at the second time point relative to the level at the first time point is indicative that the composition is efficacious in treating the metabolic disorder. After a second administration of the composition, at a third time point, a plasma sample from the individual is obtained and the level of GLP-1 and/or PYY and/or glucagon determined. An increased level of GLP-1 and/or PYY and/or decrease level of glucagon at the third time point relative to the level at the first time point is indicative that the composition is efficacious in treating the metabolic disorder. This method can be repeated for as long as it is desirable to determine whether a particular administration of the composition continues to effect an elevation in GLP-1 and/or PYY levels and/or depression in glucagon levels particularly since in some cases the dosages of the composition will need to be modified from time to time to maintain the effect of elevated GLP-1 and/or PYY and/or decreased glucagon levels in the individual.

Pharmaceutical compositions comprising a neuromedin U receptor agonist for the treatment of a metabolic disorder and those in clinical development must be administered to the individual at an appropriate dosage to be efficacious. The appropriate dosage is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can determine and prescribe the effective amount of the pharmaceutical composition required to prevent, counter or arrest the progress of the condition. Optimal precision in achieving concentrations of pharmaceutical composition within the range that yields efficacy without toxicity requires a regimen based on the kinetics of the neuromedin U receptor agonist's availability to target sites. This involves a consideration of the distribution, equilibrium, and elimination of the neuromedin U receptor agonist.

The present invention provides a novel, quantifiable method for determining the appropriate dosage of a composition comprising a neuromedin U receptor agonist that is more reliable than prior art methods. To this end, provided is a method for determining the appropriate dosage of a pharmaceutical composition comprising a neuromedin U receptor agonist given to an individual for the treatment of a metabolic disorder, comprising: (a) assaying a plasma sample from the individual to determine a level of GLP-1 and/or PYY and/or glucagon at a first time point; (b) administering the pharmaceutical composition to the individual; (c) thereafter assaying a plasma sample from the individual to determine the level of GLP-1 and/or PYY and/or glucagon at a second time point; (d) determining whether the neuromedin U receptor agonist was administered at the appropriate dosage, wherein an increased level of GLP-1 and/or PYY and/or decreased level of glucagon at the second time point relative to the first time point is indicative of the efficacy of the pharmaceutical composition in treating the metabolic disorder at the dosage administered; and (e) adjusting dosage as needed.

The methods herein may be used during clinical trials to determine promising compositions for a metabolic disorder such as diabetes or obesity therapeutics and to eliminate non-efficacious compositions from development earlier in the drug development process than by using conventional methods. Conventional measurements of efficacy such as visual analog scale assessment, questionnaires, or self-reporting may be used in conjunction with the methods herein to supplement data generated through use of the methods herein or the methods herein may be used alone.

In the methods herein, the amount of time between the first time point and the second time point defines the treatment test period being evaluated. The treatment test period can be from about two hours to about thirty days and can measured postprandially or after fasting for one to 24 hours. Currently used treatment test periods that have been used have been two hours, four hours, and 18 hours for acute peripheral administration of compositions comprising a neuromedin U receptor agonist and up to at least two weeks for chronic peripheral administration of compositions comprising a neuromedin U receptor agonist. For chronic administration, the compositions comprising a neuromedin U receptor agonist can be given once daily or in divided doses of more than one time per day. The dosing regimen may also involve once-weekly administration of the compositions comprising a neuromedin U receptor agonist or may be any dosing schedule required for the particular treatment regime.

It is not necessary for the individual to fast overnight before the commencement of the treatment test period, but the methods herein may be conducted after an overnight or longer fasting period if desired. However, it is preferred that all individuals within a single clinical trial maintain a consistent fasting period or lack thereof.

Circulating GLP-1, PYY, and glucagon are present in the plasma of rodents as well as humans. Therefore, the methods of the present invention may be performed using plasma samples from a human individual or any other animal individual in which circulating GLP-1 and/or PYY and/or glucagon can be detected in plasma. To this end, the methods herein can be used during clinical trials involving animal or human individuals to objectively determine the efficacy of the pharmaceutical composition comprising the neuromedin U receptor agonist for treating the metabolic disorder under investigation.

Detection of GLP-1, PYY, and glucagon can be by any means. Methods for detecting GLP-1, PYY, and glucagon are well known in the art. For example, immunoassays, include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin L 0 reactions, immunodiffusion assays, fluorescent immunoassays and the like. Such assays are routine and well known in the art (See, for example, Ausubel et al, eds, 1994, Current Protocols in Molecular Biology, Vol. 1, John Wiley & Sons, Inc., New York, which is incorporated by reference herein in its entirety).

Further provided are pharmaceutical compositions for elevating or enhancing GLP-1 and/or PYY levels and/or decreasing or reducing glucagon levels in an individual, in particular individuals who have a metabolic disorder in which the elevation of GLP-1 and/or PYY and the depression of glucagon in the individual is desired. Thus, pharmaceutical compositions comprising a therapeutically effective amount of one or more of the neuromedin U receptor agonists and salts thereof with a pharmaceutically acceptable carrier can be used for the treatment of a metabolic disorder in an individual in which elevating or enhancing the levels of GLP-1 and/or PYY and/or decreasing or reducing the levels of glucagon in the individual is desired. Such disorders have been defined previously but at least include, but are not limited to, obesity, metabolic syndrome or syndrome X, type II diabetes, complications of diabetes such as retinopathy, hypertension, dyslipidemias, cardiovascular disease, gallstones, osteoarthritis, and certain forms of cancers. The obesity-related disorders herein are associated with, caused by, or result from obesity.

While many treatment regimes will be designed for individuals that have a metabolic disorder, the methods herein can be used in individuals who do not have a metabolic disorder but who are in need of having their glucose levels maintained at a particular level or where hyperglycemia and/or hypoglycemia is a risk. Such individuals include those undergoing parenteral delivery of nutrients. Surgery patients, comatose patients, patients in shock, patients with gastrointestinal disease, patients with digestive hormone disease, atherosclerotic patients, patients with vascular disease, patients with liver disease, patients with liver cirrhosis, and patients with chronic pancreatitis are all examples of individuals who might need parenteral delivery of nutrients.

In general, individuals who are undergoing parenteral delivery of nutrients are at risk of developing hyperglycemia since the normal function of the alimentary route has been bypassed. In these individuals, to minimize the risk of the individual developing hyperglycemia, healthcare professionals will administer insulin to the individual during the parenteral delivery of the nutrients. However, too much insulin can cause hypoglycemia. Therefore, insulin levels have to be monitored during the parenteral administration of nutrients which is costly and time consuming and presents the risk of inducing hyperglycemia or hypoglycemia in the individual receiving the parental administration of nutrients. U.S. Pat. No. 6,852,690 discloses that exogenous administration of GLP-1 did not substantially heighten insulin release in a normal individual when the individual has a normal (non-diabetic) blood glucose level. The patent thus shows the use of GLP-1 as a substitute for insulin in parenteral administration of nutrients to an individual.

In light of the discovery herein that neuromedin U agonists effect an elevation or enhancement in GLP-1 levels in an individual, it is contemplated that neuromedin U agonists can be used in methods of parenteral administration of nutrients to an individual. The neuromedin U agonist will effect an elevation or enhancement of GLP-1 in the individual which in turn will effect an elevation in the levels of insulin in the individual. In this way, glucose levels in an individual receiving nutrients parenterally can be maintained at a normal level with reduced risk of the individual developing hyperglycemia or hypoglycemia. Thus, provided is a method of enhancing metabolism of nutrients, comprising administering by a parenteral route to a non-diabetic patient in need of enhancing metabolism of nutrients a nutritively effective amount of one or more nutrients or any combination thereof and one or more neuromedin U receptor agonists. Individuals who are especially suited for treatment include individuals with a disturbed glucose metabolism such as insulin resistance but no overt diabetes, as well as individuals who for any reason cannot receive nutrition through the alimentary canal. Such individuals include surgery patients, comatose patients, patients in shock, patients with gastrointestinal disease, patients with digestive hormone disease, and the like. In particular, obese patients, atherosclerotic patients, vascular disease patients, patients with gestational diabetes, patients with liver disease such as liver cirrhosis, patients with acromegaly, patients with glucorticoid excess such as cortisol treatment or Cushings disease, patients with activated counterregulatory hormones such as would occur after trauma, accidents and surgery and the like, patients with hypertriglyceridemia and patients with chronic pancreatitis can be readily and suitably nourished according to the invention without subjecting the patient to hypo- or hyperglycemia.

The pharmaceutical compositions may also be comprised of (in addition to neuromedin U receptor agonist and a carrier) diluents, fillers, salts, buffers, stabilizers, solubilizers, and other materials well known in the art. Compositions comprising the neuromedin U receptor agonists can be administered, if desired, in the form of salts provided the salts are pharmaceutically acceptable. Salts may be prepared using standard procedures known to those skilled in the art of synthetic organic chemistry.

“Pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to the neuromedin U receptor agonists of the general formula (I) are meant to also include the pharmaceutically acceptable salts.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially, or simultaneously.

The pharmacological composition for use in an individual to elevate the levels of GLP-1 and/or PYY and/or decrease the levels of glucagon in the individual can comprise one or more neuromedin U receptor agonists; one or more neuromedin U receptor agonists and one or more other agents for treating a metabolic disorder; or the pharmacological composition comprising the one or more neuromedin U receptor agonists can be used concurrently with a pharmacological composition comprising an agent for treating a metabolic disorder.

When the pharmacological composition comprises another agent for treating a metabolic disorder wherein the elevation or enhancement of GLP-1 and/or PYY and/or the depression or reduction of glucagon in the individual is desired or the treatment includes a second pharmacological composition comprising an agent for treating a metabolic disorder wherein the elevation or enhancement of GLP-1 and/or PYY and/or depression or reduction of glucagon in the individual is desired, the agent includes, but are not limited to, cannabinoid (CB1) receptor antagonists, glucagon like peptide 1 (GLP-1) receptor agonists, GPR119 receptor agonists, lipase inhibitors, leptin, tetrahydrolipstatin, 2-4-dinitrophenol, acarbose, sibutramine, phentamine, fat absorption blockers, simvastatin, mevastatin, ezetimibe, atorvastatin, sitagliptin, metformin, orlistat, Qnexa, topiramate, naltrexone, bupriopion, phentermine, losartan, losartan with hydrochlorothiazide, glucagon receptor antagonists, and the like. An example of a desirable composition would comprise an Neuromedin U receptor agonist and one or more dipeptidyl peptidase IV (DPP-IV) inhibitors. Examples of DPP-IV inhibitors include but are not limited to Examples of DPP-IV inhibitors include but are not limited to, isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, and SK-0403. A list of suitable agents of use in combination with a Neuromedin U receptor agonist, include, but are not limited to those compounds disclosed in Published International Application No. WO2007/109135, which is incorporated herein by reference.

Peptide-based neuromedin U receptor agonists have been described in Published International Application No. WO2007/109135, which is incorporated herein in its entirety. Briefly, these neuromedin U receptor agonists comprise the general formula (I)

Z¹-peptide-Z²

wherein the peptide has the amino acid sequence X1-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵ (SEQ ID NO:27) wherein amino acids 1 to 17 can be any amino acid or absent, wherein amino acid X¹⁸ is absent, Y, W, F, a des-amino acid or an acyl group; amino acid X¹⁹ is A, W, Y, F or an aliphatic amino acid; amino acid X²⁰ is absent, L, G, sarcosine (Sar), D-Leu, NMe-Leu, D-Ala or A; amino acid X²¹ is F, NMe-Phe, an aliphatic amino acid, an aromatic amino acid, A or W; X²² is R, K, A or L; amino acid X²³ is P, Sar, A or L; amino acid X²⁴ is R, Harg or K; and amino acid X²⁵ is N, any D- or L-amino acid, Nle or D-Nle, A; and Z¹ is an optionally present protecting group that, if present, is joined to the N-terminal amino group; and Z² is NH2 or an optionally present protecting group that, if present, is joined to the C-terminal carboxy group, and pharmaceutically acceptable salts thereof.

In particular embodiments, the peptide comprises the amino acid sequence X¹-X²-X³-X⁴-X⁵-X⁶-X⁷-X⁸-X⁹-X¹⁰-X¹¹-X¹²-X¹³-X¹⁴-X¹⁵-X¹⁶-X¹⁷-X¹⁸-F-L-F-R-P-R-N (SEQ ID NO:1) wherein amino acids 1 to 17 can be any amino acid or absent. The amino acid sequences of particular neuromedin U receptor agonists having the above amino acid sequence are shown in Table 1.

In further embodiments, the peptide comprises the amino acid sequence F-R-V-D-E-E-F-Q-S-P-F-A-S-Q-S—R-G-X18-X19-X20-X21-X22-X23-X24-X25 (SEQ ID NO:7) wherein amino acid X¹⁸ is absent, Y, W, F, a des-amino acid or an acyl group; amino acid X¹⁹ is A, W, Y, F or an aliphatic amino acid; amino acid X²⁰ is absent, G, sarcosine (Sar), D-Leu, NMe-Leu, D-Ala or A; amino acid X²¹ is NMe-Phe, an aliphatic amino acid, an aromatic amino acid, A or W; amino acid X²² is K, A or L; amino acid X²³ is Sar, A or L; amino acid X²⁴ is Harg or K; and amino acid X²⁵ is any D- or L-amino acid, Nle or D-Nle, or A. Examples of peptides having the above amino acid sequence are shown in Table 1

In yet another embodiment, the peptide comprises the amino acid sequence X¹-X²-X³-X⁴-X⁵-X⁶-X⁷-X⁸ (SEQ ID NO:8) wherein amino acid X¹ is absent, Y, W, F, a des-amino acid or an acyl group; amino acid X² is A, W, Y, F or an aliphatic amino acid; amino acid X³ is absent, G, sarcosine (Sar), D-Leu, NMe-Leu, D-Ala or A; amino acid X⁴ is NMe-Phe, an aliphatic amino acid, an aromatic amino acid, A or W; amino acid X⁵ is K, A or L; amino acid X⁶ is Sar, A or L; amino acid X¹ is Harg or K; and amino acid X⁸ is any D- or L-amino acid, Nle or D-Nle, or A. Examples of peptides having the above amino acid sequence are shown in Table 2.

In particular aspects, the neuromedin U receptor agonist optionally includes a protecting group covalently joined to the N-terminal amino group. A protecting group covalently joined to the N-terminal amino group of the neuromedin U receptor agonists reduces the reactivity of the amino terminus under in vivo conditions. Amino protecting groups include —C₁₋₁₀ alkyl, —C₁₋₁₀ substituted alkyl, —C₂₋₁₀ alkenyl, —C₂₋₁₀ substituted alkenyl, aryl, —C₁₋₆ alkyl aryl, —C(O)—(CH₂)₁₋₆—COOH, —C(O)—C₁₋₆ alkyl, —C(O)-aryl, —C(O)—O—C₁₋₆ alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. Deamination of the N-terminal amino acid is another modification that is contemplated for reducing the reactivity of the amino terminus under in viva conditions.

Chemically modified compositions of the neuromedin U receptor agonists wherein the neuromedin U receptor agonist derivatives are linked to a polymer are also included within the scope of the present invention. The polymer selected is usually modified to have a single reactive group, such as an active ester for acylation or an aldehyde for alkylation, so that the degree of polymerization may be controlled as provided for in the present methods. Included within the scope of polymers is a mixture of polymers. Preferably, for therapeutic use of the end-product preparation, the polymer will be pharmaceutically acceptable.

The polymer or mixture thereof may be selected from the group consisting of; for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the neuromedin U receptor agonists are modified by PEGylation, cholesteroylation, or palmitoylation. The modification can be to any amino acid residue in the neuromedin U receptor agonist, however, in currently preferred embodiments, the modification is to the N-terminal amino acid of the neuromedin U receptor agonist, either directly to the N-terminal amino acid or by way coupling to the thiol group of a cysteine residue added to the N-terminus or a linker added to the N-terminus such as Ttds. In further embodiments, the N-terminus of the neuromedin U receptor agonist comprises a cysteine residue to which a protecting group is coupled to the N-terminal amino group of the cysteine residue and the cysteine thiolate group is derivatized with N-ethylmaleimide, PEG group, cholesterol group, or palmitoyl group. In further still embodiments, an acetylated cysteine residue is added to the N-terminus of the neuromedin U receptor agonists, and the thiol group of the cysteine is derivatized with N-ethylmaleimide, PEG group, cholesterol group, or palmitoyl group.

The neuromedin U receptor agonist can comprise other non-sequence modifications, for example, glycosylation, lipidation, acetylation, phosphorylation, carboxylation, methylation, or any other manipulation or modification, such as conjugation with a labeling component. While, in particular aspects, the neuromedin U receptor agonist herein utilize naturally-occurring amino acids or D isoforms of naturally occurring amino acids, substitutions with non-naturally occurring amino acids (for example., methionine sulfoxide, methionine methylsulfonium, norleucine, epsilon-aminocaproic acid, 4-aminobutanoic acid, tetrahydroisoquinoline-3-carboxylic acid, 8-aminocaprylic acid, 4 aminobutyric acid, Lys(N(epsilon)-trifluoroacetyl) or synthetic analogs, for example, o-aminoisobutyric acid, p or y-amino acids, and cyclic analogs. In further still aspects, the neuromedin U receptor agonists comprise a fusion protein that having a first moiety, which is a neuromedin U receptor agonist, and a second moiety, which is a heterologous peptide.

The neuromedin U receptor agonist may be modified by a variety of chemical techniques to produce derivatives having essentially the same activity as the unmodified neuromedin U receptor agonist and/or having other desirable properties. A protecting group covalently joined to the C-terminal carboxy group reduces the reactivity of the carboxy terminus under in vivo conditions. For example, carboxylic acid groups of the peptide, whether carboxyl-terminal or side chain, may be provided in the form of a salt of a pharmacologically-acceptable cation or esterified to form a C1-6 ester, or converted to an amide of formula NRR2 wherein R and R2 are each independently H or C1-6 alkyl, or combined to form a heterocyclic ring, such as a 5- or 6-membered ring. The carboxy terminus protecting group is preferably attached to the α-carbonyl group of the last amino acid. Carboxy terminus protecting groups include, but are not limited to, amide, methylamide, and ethylamide. Amino groups of the peptide, whether N-terminal or side chain, may be in the form of a pharmacologically-acceptable acid addition salt, such as the HCl, HBr, acetic, benzoic, toluene sulfonic, maleic, tartaric, and other organic salts, or may be modified to C₁₋₆ alkyl or dialkyl amino or further converted to an amide.

Hydroxyl groups of the neuromedin U receptor agonist side chain may be converted to C₁₋₆ alkoxy or to a C₁₋₆ ester using well-recognized techniques. Phenyl and phenolic rings of the peptide side chain may be substituted with one or more halogen atoms, such as fluorine, chlorine, bromine or iodine, or with C₁₋₆ alkyl, C₁₋₆ alkoxy, carboxylic acids and esters thereof, or amides of such carboxylic acids. Methylene groups of the neuromedin U receptor agonist side chains can be extended to homologous C₂₋₄ alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability. For example, a carboxyl-terminal or amino-terminal cysteine residue can be added to the peptide, so that when oxidized the peptide will contain a disulfide bond, thereby generating a cyclic peptide. Other peptide cyclizing methods include the formation of thioethers and carboxyl- and amino-terminal amides and esters.

Polysaccharide polymers are another type of water soluble polymer that may be used for protein modification. Dextrans are polysaccharide polymers comprised of individual subunits of glucose predominantly linked by α1-6 linkages. The dextran itself is available in many molecular weight ranges, and is readily available in molecular weights from about 1 kDa to about 70 kDa. Dextran is a suitable water soluble polymer for use as a vehicle by itself or in combination with another vehicle (See, for example, WO96/11953 and WO96/05309). The use of dextran conjugated to therapeutic or diagnostic immunoglobulins has been reported; see, for example, European Patent Publication No. 0 315 456. Dextran of about 1 kDa to about 20 kDa is preferred when dextran is used as a vehicle in accordance with the present invention.

Examples of neuromedin U receptor agonists are shown in Table 1.

TABLE 1

| SEQ | | |
|---|---|---|
| ID | | Neuromedin U Receptor Agonist |
| NO. | Peptides | Sequences |
| 2 | NMU | FRVDEEFQSPFASQSRGYFLFRPRN-CONH₂ |
| 3 | NMU1 | P₁-FRVDEEFQSPFASQSRGYFLFRPRN-CONH₂ |
| 4 | NMU2 | Ac-FRVDEEFQSPFASQSRGYFLFRPRN-CONH₂ |
| 5 | NMU3 | GYFLFRPRN-CONH₂ |
| 28 | NMU4 | FRVDEEFQSPFASQSRPYFLFRPRN-CONH₂ |
| 6 | NMU5 | Ac-FRVDEEFQSPFASQSRPYFLFRPRN-CONH₂ |
| 29 | Pre-1 | Ac-CGYFLFRPRN-CONH₂ |
| 30 | NMU6 | Ac-C1GYFLFRPRN-CONH₂ |
| 31 | NMU7 | Ac-C₂GYFLFRPRN-CONH₂ |
| 32 | NMU11 | Ac-C₃GYFLFRPRN-CONH₂ |
| 33 | Pre-2 | Ac-CFRVDEEFQSPFASQSRGYFLFRPRN-CONH₂ |
| 34 | NMU8 | Ac-C₁FRVDEEFQSPFASQSRGYFLFRPRN-CONH₂ |
| 35 | NMU9 | Ac-C₂FRVDEEFQSPFASQSRGYFLFRPRN-CONH₂ |
| 36 | NMU12 | Ac-C₂FRVDEEFQSPFASQSRGYFLFRPRN-CONH₂ |
| 37 | NMU10 | Ac-C₃FRVDEEFQSPFASQSRGYFLFRPRN-CONH₂ |
| 38 | NMU21 | Ac-C₄FRVDEEFQSPFASQSRGYFLFRPRN-CONH₂ |
| 39 | NMU26 | Ac-C₅FRVDEEFQSPFASQSRGYFLFRPRN-CONH₂ |
| 25 | Pre-3 | Ac-CFRVDEEFQSPFASQSRGYFaWRPRN-CONH₂ |
| 40 | NMU13 | Ac-C₁FRVDEEFQSPFASQSRGYFaWRPRN-CONH₂ |
| 41 | NMU14 | Ac-C₂FRVDEEFQSPFASQSRGYFaWRPRN-CONH₂ |
| 42 | Pre-4 | Ac-C-Ttds-FLFRPRN-CONH₂ |
| 43 | NMU15 | Ac-C₁-Ttds-FLFRPRN-CONH₂ |
| 44 | NMU16 | Ac-C₂-Ttds-FLFRPRN-CONH₂ |
| 45 | Pre-5 | Ac-CASQSRGYFLFRPRN-CONH₂ |
| 46 | NMU17 | Ac-C₁ASQSRGYFLFRPRN-CONH₂ |
| 47 | NMU18 | Ac-C₂ASQSRGYFLFRPRN-CONH₂ |
| 48 | Pre-6 | Ac-CFQSPFASQSRGYFLFRPRN-CONH₂ |
| 49 | NMU19 | Ac-C₁FQSPFASQSRGYFLFRPRN-CONH₂ |
| 50 | NMU20 | Ac-C₂FQSPFASQSRGYFLFRPRN-CONH₂ |
| 51 | Pre-7 | Ac-C-C-FRVDEEFQSPFASQSRGYFLFRPRN-CONH₂ |
| 52 | NMU22 | Ac-C₁-C₁-FRVDEEFQSPFASQSRGYFLFRPRN- |
| | | CONH₂ |
| 53 | NMU23 | Ac-C₄-C₄-FRVDEEFQSPFASQSRGYFLFRPRN- |
| | | CONH₂ |
| 54 | Pre-8 | Pam-CFRVDEEFQSPFASQSRGYFLFRPRN-CONH2 |
| 55 | NMU24 | Pam-C₁FRVDEEFQSPFASQSRGYFLFRPRN-CONH2 |
| 56 | NMU25 | Pam-C₂FRVDEEFQSPFASQSRGYFLFRPRN-CONH2 |
| 57 | NMU27 | Ac-C₆FRVDEEFQSPFASQSRGYFLFRPRN-CONH2 |
| 58 | Pre-9 | Ac-C-Ttds-GYFLFRPRN-CONH2 |
| 59 | NMU28 | Ac-C₁-Ttds-GYFLFRPRN-CONH₂ |
| 60 | NMU29 | Ac-C₂-Ttds-GYFLFRPRN-CONH₂ |
| 61 | NMU30 | Ac-C1FRVDEEFQSPFASQSRPYFLFRPRN-CONH₂ |
| 62 | NMU31 | Ac-C2FRVDEEFQSPFASQSRPYFLFRPRN-CONH₂ |

C = cysteine; P₁ = (PEG)₂40 kDa; C₁ = Cys(N-ethylmaleimidyl), C₂ = Cys(PEG)₂40 kDa, C₄ = Cys(PEG)20 kDa, C₅ = Cys(PEG)220 kDa, C₆ = Cys(PEG)40 kDa each corresponding to a cysteine residue PEGylated via the side-chain thiol with a branched PEG [(PEG)₂] or a linear PEG of the indicated MW; C₃ = Cys(Cholesteroyl), corresponding to a cysteine residue linked to cholesterol via the side-chain thiol; Ttds, 1-amino-4,7,10-trioxa-13-tridecanamine succinimic acid; a, D-Alanine; Ac = acetyl; Pam = palmitoyl

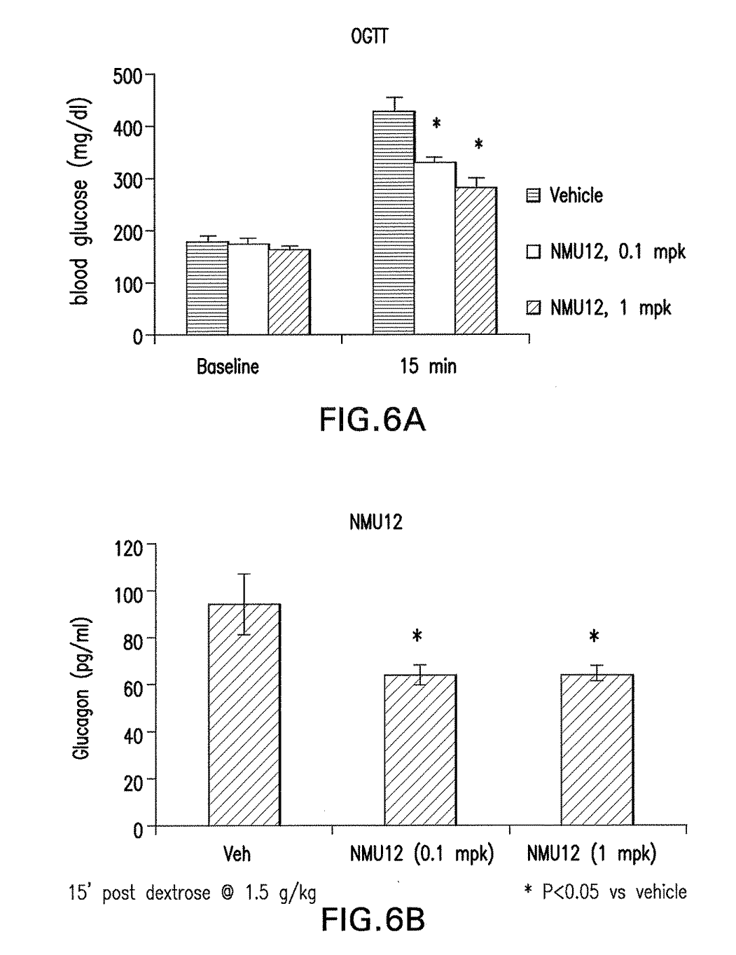

The neuromedin U receptor agonists shown in Table 1, with the exception of those neuromedin U receptor agonists comprising the amino acid sequence of SEQ ID NO:25, are bispecific in that they can bind and activate either NMUR1 or NMUR2 receptors. The neuromedin U receptor agonists comprising SEQ ID NO:25 have been found to be NMUR1 specific. PEGylation of the neuromedin U receptor agonists shown in Table 1 appear to extend the serum half-life of the neuromedin U receptor agonists and significantly, render particular neuromedin U receptor agonists such as NMU 12 to be capable of crossing the blood-brain barrier. For example, as shown in Example 4 and FIGS. 6A and 6B, NMU12 was shown to be able to reduce food intake and reduce weight gain in Nmur1 knockout mice. The results indicate that PEGylated peptide NMU12 administered peripherally was able to cross the blood-brain barrier. PEGylation appeared to extend the serum half-life of NMU1, NMU9, and NMU20 by three days and NMU11 and NMU18 by two days. NMU9 and NMU12 differ by the source of (PEG) 240 kDa covalently joined to the thiol group of the N-terminal cysteine residue.

Examples of neuromedin U receptor agonists comprising the amino acid sequence F-R-V-D-E-E-F-Q-S-P-F-A-S-Q-S-R-G-X¹⁸-X¹⁹-X²⁰-X²¹-X²²-X²³-X²⁴-X²⁵ (SEQ ID NO:7) or X¹—X²-X³-X⁴-X⁵-X⁶-X⁷-X⁸ (SEQ ID NO:8) wherein amino acid X¹⁸ or X¹ is absent, Y, W, F, a des-amino acid or an acyl group; amino acid X¹⁹ or X² is A, W, Y, F or an aliphatic amino acid; amino acid X²⁰ or X³ is absent, G, sarcosine (Sar), D-Leu, NMe-Leu, D-Ala or A; amino acid X²¹ or X⁴ is NMe-Phe, an aliphatic amino acid, an aromatic amino acid, A or W; amino acid X²² or X⁵ is K, A or L; amino acid X²³ or X⁶ is Sar, A or L; amino acid X²⁴ or X⁷ is Harg or K; and amino acid X²⁵ or X⁸ is any D- or L-amino acid, Nle or D-Nle, or A. Examples of peptides having the above amino acid sequence are shown in Table 2. In general, the peptides comprising SEQ ID NO:7 or SEQ ID NO:8 are specific for NMUR1 receptor; however, as shown in the Examples, neuromedin U receptor agonists N, O, and P are bispecific.

TABLE 2

| SEQ | | |
|---|---|---|
| ID | | Neuromedin U Receptor |
| NO. | Peptides | Agonist Sequences |
| 9 | A | YFWRPRN-CONH₂ |
| 10 | B | YF-(D-L)-WRPRN-CONH₂ |
| 11 | C | YFGWRPRN-CONH₂ |
| 12 | D | YF-(D-A)-WRPRN-CONH₂ |
| 26 | E | Ac-F-(D-L)-WRPRN-CONH₂ |
| 13 | F | Ac-FFRPRN-CONH₂ |
| 14 | G | FRVDEEFQSPFASQSRGYFWRPRN-CONH₂ |
| 15 | H | FRVDEEFQSPFASQSRGYF-(D-L)-WRPRN-CONH₂ |
| 16 | I | FWLFRP-(Harg)-N-CONH₂ |
| 17 | J | FWLFRA-(Harg)-N-CONH₂ |
| 18 | K | WFLFRAR-(D-Nle)-CONH₂ |
| 19 | L | FWLFRARN-CONH₂ |
| 20 | M | WALFRARN-CONH₂ |
| 21 | N | FALFRPRN-CONH₂ |
| 22 | O | FRVDEEFQSPFASQSRGFWLFRP-(Harg)-N-CONH₂ |
| 23 | P | FRVDEEFQSPFASQSRGFWLFRA-(Harg)-N-CONH₂ |
| 24 | Q | FRVDEEFQSPFASQSRGFWLFRPR-(D-Nle)-CONH₂ |

Methods of administrating the pharmacological compositions for elevating GLP-1 and/or PYY and/or decreasing glucagon levels in an individual include, but are not limited to, intradermal, intramuscular, intraperitoneal, intravenous, subcutaneous, intranasal, epidural, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Intraventricular injection may be facilitated by an intraventricular catheter attached to a reservoir (for example, an Ommaya reservoir). Pulmonary administration may also be employed by use of an inhaler or nebulizer, and formulation with an aerosolizing agent. It may also be desirable to administer the composition locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the compositions for elevating the GLP-1 and/or PYY and/or decreasing glucagon levels in an individual including, but not limited to, encapsulation in liposomes, microparticles, microcapsules; minicells; polymers; capsules; tablets; and the like. In one embodiment, the composition may be delivered in a vesicle, in particular a liposome. In a liposome, the composition is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028 and U.S. Pat. No. 4,737,323. In yet another embodiment, the composition can be delivered in a controlled release system including, but not limited to: a delivery pump (See, for example, Saudek, et al., New Engl. T. Med. 321: 574 (1989) and a semi-permeable polymeric material (See, for example, Howard, et al., J. Neurosurg. 71: 105 (1989)). Additionally, the controlled release system can be placed in proximity of the therapeutic target (for example, the brain), thus requiring only a fraction of the systemic dose. See, for example, Goodson, In: Medical Applications of Controlled Release, 1984. (CRC Press, Bocca Raton, Fla.).

The amount of the composition that will be effective in elevating the GLP-1 and/or PYY and/or decreasing glucagon levels in an individual will depend on the nature of the disorder or condition afflicting the individual and may be determined by standard clinical techniques by those of average skill within the art. The methods disclosed herein are used to help identify optimal dosage ranges. The precise dose to be employed in the formulation will also depend on the route of administration, and the overall seriousness of the disease or disorder, and should be decided according to the judgment of the practitioner and each individual's circumstances. Ultimately, the attending physician will decide the amount of the composition with which to treat each individual patient based on the methods disclosed herein. Initially, the attending physician will administer low doses of the composition and measure the level of GLP-1 and/or PYY and/or glucagon in the plasma of the individual two or more hours post-administration. The attending physician will then compare the level to the level in the individual prior to administration of the dose. Larger doses of the composition may be administered until the optimal level of GLP-1 and/or PYY and/or glucagon is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. However, suitable dosage ranges for intravenous administration of the compositions are generally about 5-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using the compositions. Dosage will also vary according to the age, weight and response of the individual patient.

The following examples are intended to promote a further understanding of the present invention.

### Example 1

This example shows that both a wild-type, native NMU and a PEGylated NMU analog (NMU12) were effective in elevating the basal levels of PYY and GLP-1 in mice and reducing glucagon levels in the mice during a glucose tolerance test. These peptides are shown in Table 1.

Animal dosing was as follows. Ad libitum fed male diet-induced obese mice were weighed and dosed subcutaneously about 30 minutes prior to the onset of the dark phase of the light cycle. Animals were either not given food or allowed food during treatment. For NMU studies, plasma was collected by cardiac sticks at two or four hours post dose. For studies with the PEGylated NMU analog, plasma was collected at multiple time points up to 18-hours post dose.

The Oral Glucose Tolerance Test (OGTT) was performed as follows: Ad libitum fed male diet-induced obese mice were weighed and dosed subcutaneously about 30 minutes prior to the onset of the dark phase of the light cycle. Animals were not given food after dosing. The next day, approximately 18-hours after dosing, basal glucose levels were measured from animals by tail blood. Animals were then administered a glucose dose (1.5 g/kg) by oral gavage and fifteen minutes after dosing with glucose, plasma was collected to evaluate glucagon and glucose levels.

GLP-1 levels in plasma were measured using the Total GLP-1 kit from Meso Scale Discovery, Gaithersburg, Md. Glucagon levels were measured using the Metabolic Multiplex kit from Meso Scale Discovery, Gaithersburg, Md. PYY levels were measured using the PYY Luminex assay from Millipore, Billerica, Mass. The data shown reports of glucagon, PYY and GLP-1 levels as pg/mL (all values are reported as mean±SEM and data was analyzed using a two-tailed unpaired Student's t test; p values ≦0.05 were reported as significant and are denoted with an asterisk).

Diet-induced obese mice (DIO) were purchased from Taconic Farms. NMUR1 knockout (Nmur1−/−) mice were generated using standard homologous recombination techniques. U.S. Published Application No. 20080172752 discloses NMUR1 knockout mice. NMUR2 knockout (Nmur2−/−) mice were licensed from Deltagen Inc., San Mateo, Calif. and subsequently transferred to Taconic Farms. Mice were individually housed in Tecniplast cages in a conventional SPF facility. Mice were initially maintained on a regular chow diet and then early in their life were switched to a high fat diet (D12492: 60% kcal from fat; Research Diets, Inc., New Brunswick, N.J.) with ad libitum access to water in a 12-hour light/12-hour dark cycle.

The results of the animal dosing shows that both NMU and NMU analogs effect an increase the serum levels of PYY and GLP-1 in the mice over time.

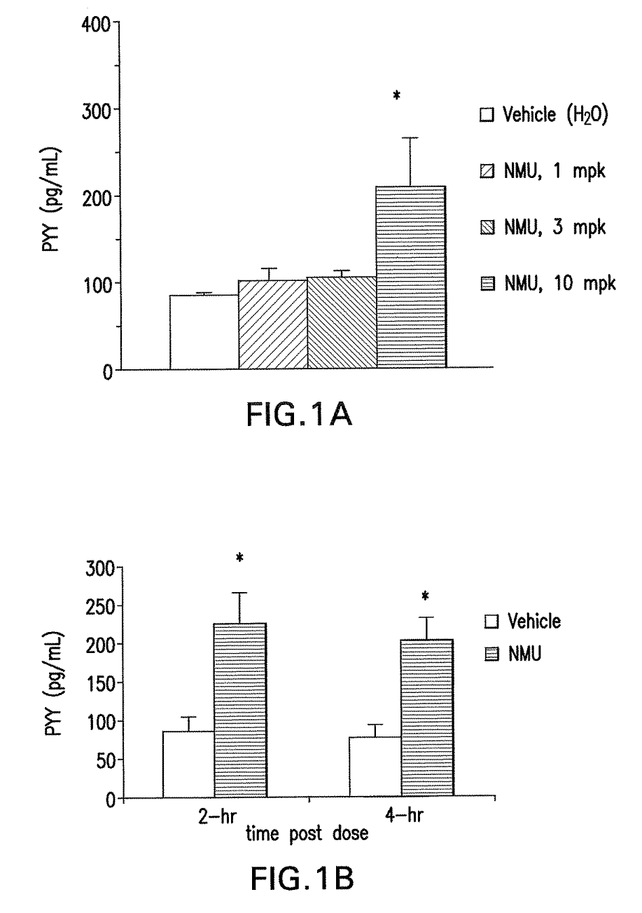

Acute peripheral administration of NMU increased plasma levels of PYY in obese mice. FIG. 1A shows that 10 mpk of NMU increased PYY levels at four-hours post dose by about two-fold. FIG. 1B shows that 10 mpk of NMU increased PYY levels at both two-hours and four-hours post dose.

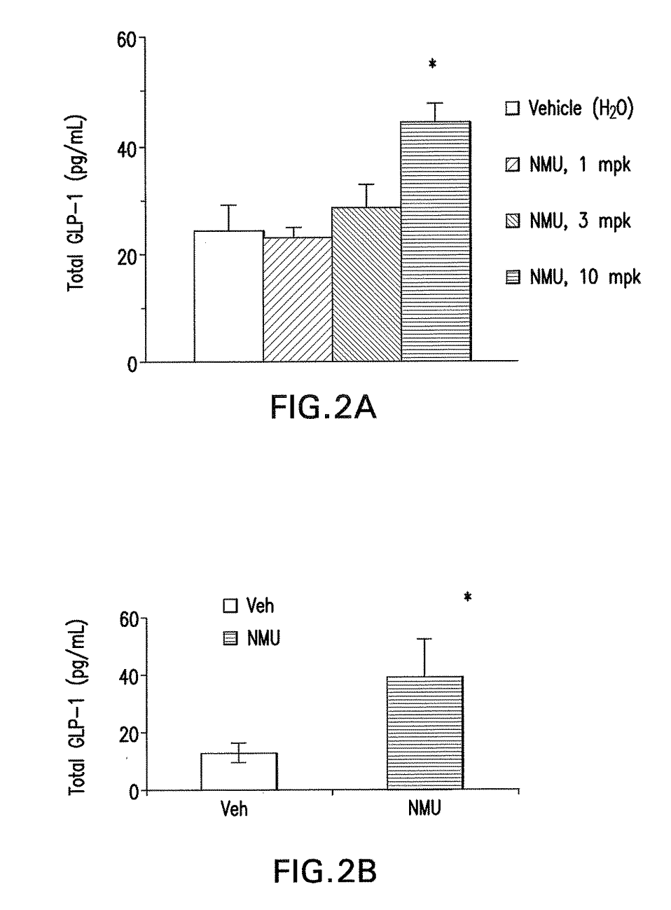

Acute peripheral administration of NMU increased plasma levels of total GLP-1 in obese mice. FIG. 2A shows that 10 mpk of NMU increased GLP-1 levels at four-hours post dose. FIG. 2B shows that chronic treatment of NMU (3 mpk/day) given subcutaneously for two weeks led to an increase in total GLP-1.

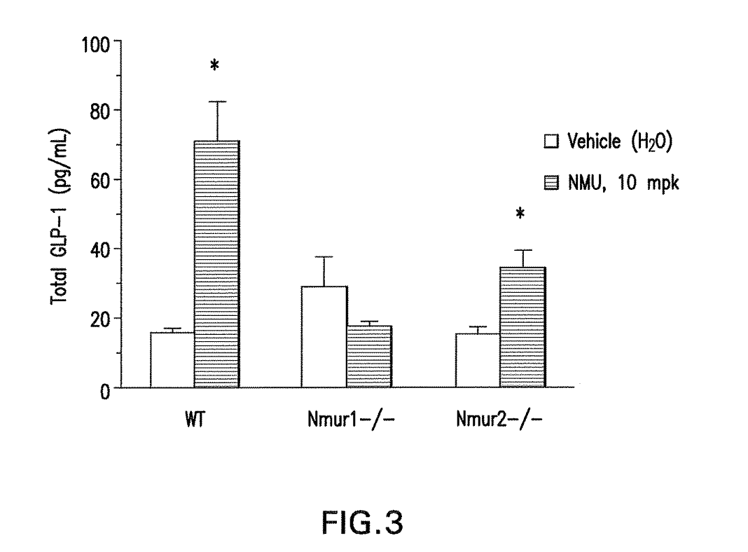

The increase in GLP-1 levels are mediated by NMUR1 as shown in FIG. 3. Plasma was collected from wild-type, Nmur1-deficient and Nmur2-deficient mice four-hours post dose in the absence of food. Increased levels of GLP-1 were observed in wild-type and Nmur2-deficient mice but not in Nmur-1 deficient mice. This result indicates that NMUR1 is in involved in the observed increase of plasma GLP-1 levels.

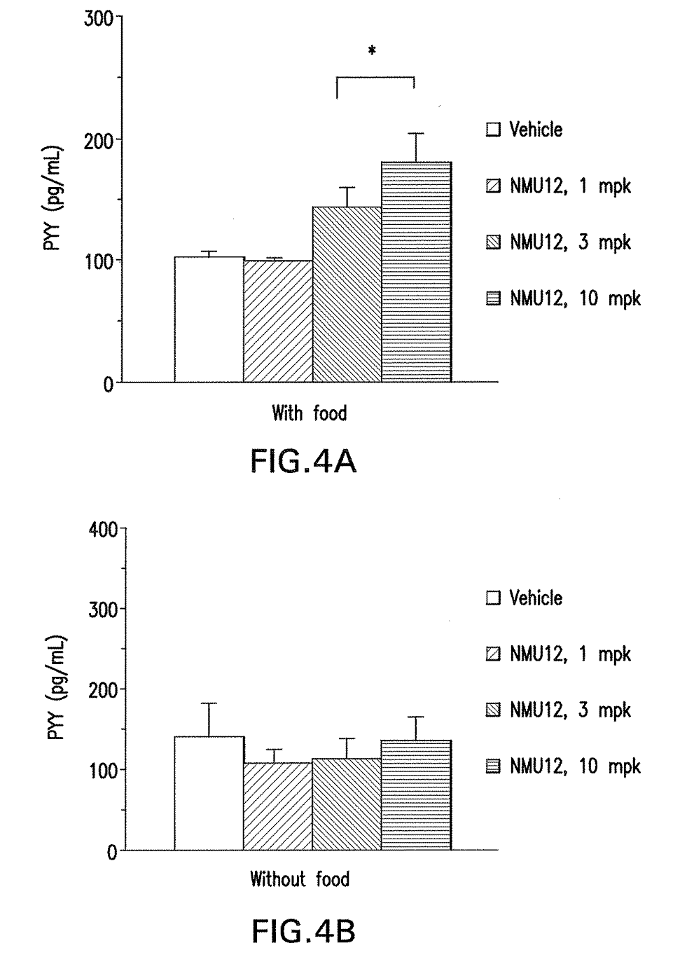

Acute peripheral administration of a PEGylated NMU analog increases plasma levels of PYY in obese mice. FIG. 4A shows that the PEGylated NMU analog dose-dependently increased PYY levels in plasma 18-hours post dose in the presence of food. However, as shown in FIG. 48, in the absence of food, 10 mpk the PEGylated NMU analog did not increase PYY levels 18-hr post dose.

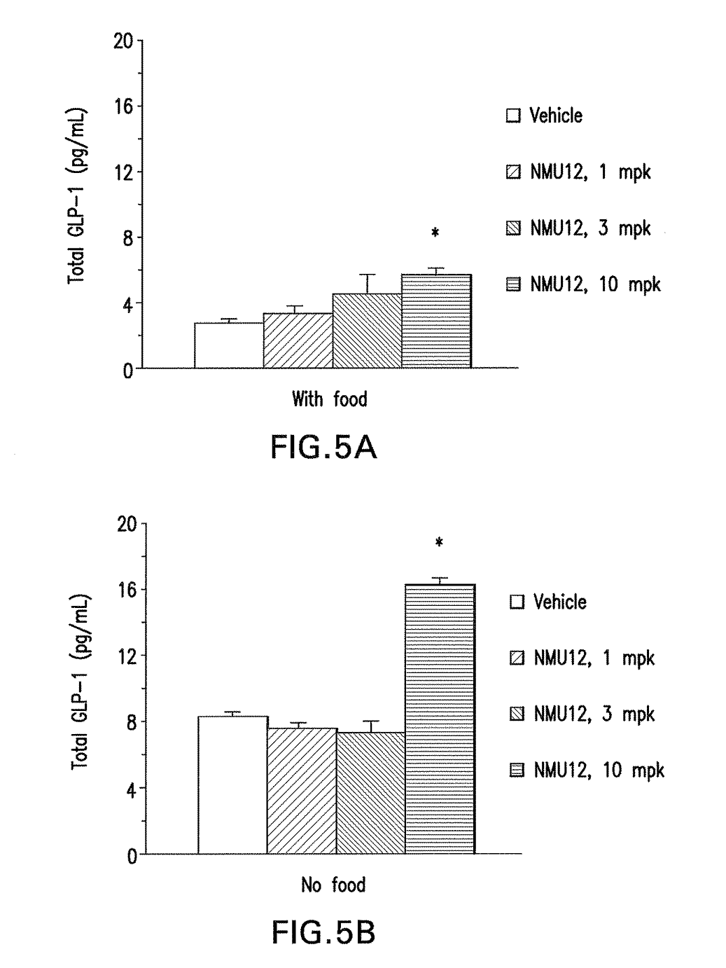

Acute peripheral administration of the PEGylated NMU analog increased plasma levels of total GLP-1 in obese mice. FIG. 5A shows that 10 mpk of the PEGylated NMU analog increased GLP-1 levels at 18-hours post dose in the presence of food. However, in contrast to the effect observed for PYY, FIG. 5B shows that the PEGylated NMU analog also effected an increase in GLP-1 levels in the absence of food.

Acute peripheral administration of the PEGylated NMU analog improved glucose excursion during an oral glucose tolerance test as shown in FIG. 6A. Additionally, acute peripheral administration of the PEGylated NMU analog reduced plasma levels of glucagon in obese mice during an OGTT as shown in FIG. 6B.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method of determining the efficacy of a composition comprising a neuromedin U receptor agonist given to an individual for the treatment of a metabolic disorder, comprising:
(a) assaying a plasma sample from the individual to determine a level of glucagon-like peptide 1 (GLP-1) and/or peptide YY (PYY) at a first time point;
(b) administering the composition to the individual; and
(c) thereafter assaying a plasma sample from the individual to determine the level of GLP-1 and/or PYY at a second time point;
wherein an increased level of GLP-1 and/or PYY at the second time point relative to the first time point is indicative of the efficacy of the composition in treating the metabolic disorder.

2. The method of claim 1, wherein the individual is a human.

3. The method of claim 1, wherein the individual is a rodent.

4. The method of claim 3, wherein the individual is a primate.

5. The method of claim 1, wherein the level of GLP-1 and/or PYY is determined by radioimmunoassay.

6. The method of claim 1, wherein the level of GLP-1 and/or PYY is determined by ELISA.

7. The method of claim 1, wherein the level of GLP-1 and/or PYY is determined by radioligand binding assay.

8. The method of claim 1, wherein the level of GLP-1 and/or PYY is determined by liquid chromatography.

9. The method of claim 1, wherein the amount of time between the first time point and the second time point is at least two hours.

10-11. (canceled)

12. A method for elevating or enhancing glucagon-like peptide 1 (GLP-1) and/or peptide YY (PYY) levels in an individual comprising administering to the individual a composition comprising a neuromedin U receptor agonist, which has the formula
Z1-peptide-Z²
wherein the peptide has the amino acid sequence X1-X2-X3-X4-X5-X6-X7-X8-X9-X10-X11-X12-X13-X14-X15-X16-X17-X18-X19-X20-X21-X22-X23-X24-X25 (SEQ ID NO:27), wherein amino acids 1 to 17 can be any amino acid or absent; wherein amino acid X18 is absent, Y, W, F, a des-amino acid or an acyl group; amino acid X19 is A, W, Y, F or an aliphatic amino acid; amino acid X20 is absent, L, G, sarcosine (Sar), D-Leu, NMe-Leu, D-Ala or A; amino acid X21 is F, NMe-Phe, an aliphatic amino acid, an aromatic amino acid, A or W; X22 is R, K, A or L; amino acid X23 is P, Sat, A or L; amino acid X24 is R, Harg or K; and amino acid X25 is N, any D- or L-amino acid, Nle or D-Nle, A.; and Z1 is an optionally present protecting group that, if present, is joined to the N-terminal amino group; and Z2 is NH2 or an optionally present protecting group that, if present, is joined to the C-terminal carboxy group, and pharmaceutically acceptable salts thereof.

13. The method of claim 12, wherein the composition further includes one or more dipeptidyl peptidase TV (DPP-IV) inhibitors and pharmaceutically acceptable salts thereof.

14. The method of claim 13, wherein the DPP-IV inhibitor is isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, or SK-0403.

15. The use of the composition of claim 12 in the preparation of a medicament for elevating the level of GLP-1 and/or PYY in an individual.

16. (canceled)

17. A method for elevating or enhancing glucagon-like peptide 1 (GLP-1) and/or peptide YY (PYY) levels and decreasing or reducing glucagon levels in an individual comprising administering to the individual a composition comprising a neuromedin U receptor agonist, which has the formula
Z1-peptide-Z²
wherein the peptide has the amino acid sequence X1-X2-X3-X4-X5-X6-X7-X8-X9-X10-X11-X12-X13-X14-X15-X16-X17-X18-X19-X20-X21-X22-X23-X24-X25 (SEQ ID NO:27), wherein amino acids 1 to 17 can be any amino acid or absent; wherein amino acid X18 is absent, Y, W, F, a des-amino acid or an acyl group; amino acid X19 is A, W, Y, F or an aliphatic amino acid; amino acid X20 is absent, L, G, sarcosine (Sar), D-Leu, NMe-Leu, D-Ala or A; amino acid X21 is F, NMe-Phe, an aliphatic amino acid, an aromatic amino acid, A or W; X22 is R, K, A or L; amino acid X23 is P, Sar, A or L; amino acid X24 is R, Harg or K; and amino acid X25 is N, any D- or L-amino acid, Nle or D-Nle, A.; and Z1 is an optionally present protecting group that, if present, is joined to the N-terminal amino group; and Z2 is NH2 or an optionally present protecting group that, if present, is joined to the C-terminal carboxy group, and pharmaceutically acceptable salts thereof.

18. The method of claim 17, wherein the composition further includes one or more dipeptidyl peptidase IV (DPP-IV) inhibitors and pharmaceutically acceptable salts thereof.

19. The method of claim 18, wherein the DPP-IV inhibitor is isoleucine thiazolidide, valine pyrrolidide, sitagliptin, saxagliptin, NVP-DPP728, LAF237 (vildagliptin), P93/01, TSL 225, TMC-2A/2B/2C, FE 999011, P9310/K364, VIP 0177, SDZ 274-444, GSK 823093, E 3024, SYR 322, TS021, SSR 162369, GRC 8200, K579, NN7201, CR 14023, PHX 1004, PHX 1149, PT-630, or SK-0403.

20. (canceled)
